SEQUENCE LISTING (2 sequences)

SEQ ID NO: 1 - protein, 4536 aa, Homo sapiens
EEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYNNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPETDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVVENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELTHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMQNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 2 - protein, 10 aa, Homo sapiens
VPSCKLDFRE

(19) United States
(12) Patent Application Publication
(10) Pub. No.: US 2009/0136985 A1
(43) Pub. Date: May 28, 2009

(54) Vascular Aging-Predicting Factor and Utilization of the Same

(76) Inventors: Takahito Kondo, Nagasaki (JP); Yoshito Ihara, Nagasaki (JP); Yoshishige Urata, Nagasaki (JP); Shinji Goto, Nagasaki (JP); Takaaki Kono, Nagasaki (JP)

Correspondence Address: WENDEROTH, LIND & PONACK, L.L.P., 1030 15th Street, N.W.,, Suite 400 East, Washington, DC, 20005-1503, US

(21) Appl. No.: 12/085,656

(22) PCT Filed: Oct. 30, 2006
(86) PCT No.: PCT/JP2006/321610

§ 371 Date: Aug. 28, 2008

(30) Foreign Application Priority Data
Nov. 29, 2005 (JP) 2005-344630

(51) Int. Cl. C12Q 1/02; C07K 7/06; C07K 16/18

(52) U.S. Cl. 435/29; 530/328; 530/389.8; 530/387.9

(57) ABSTRACT

Abstract A predicting factor for vascular aging and an examination method for an early-stage lesion resulting from vascular aging are provided. Specifically, a predicting factor for vascular aging comprising a human apolipoprotein B100 having a glutathionylated thiol group; a method of examining a lesion resulting from vascular aging contain measurement of a human apolipoprotein B100 having a glutathionylated thiol group in the blood sample; an antibody specifically recognizing an apolipoprotein B100 having a glutathionylated thiol group; a diagnostic agent or a diagnostic kit for an early-stage lesion resulting from vascular aging containing an antibody recognizing a human apolipoprotein B100 having a glutathionylated thiol group, are provided.

### TECHNICAL FIELD

The present invention relates to a predicting factor for vascular aging and utilization thereof. In particular, the present invention relates to a predicting factor for vascular aging using glutathionylation of a specific thiol group of a human apolipoprotein B100 as an index and a detection method thereof.

### BACKGROUND ART

In the metabolic syndrome which is expected to spread more and more in the future, diabetes mellitus (impaired glucose tolerance), hypertension, hyperlipidemia, obesity, and the like which are underlying diseases for the metabolic syndrome act commonly as a promoting factor for vascular aging. Vascular aging causes cardiac infarction and cerebral infarction through development of arterial sclerosis, arterial occlusion or the like. However, objective indices available for the vascular aging are those obtained by morphological measurements such as ultrasonic diagnostics of the carotid artery, and a simple measurement method using serum of a patient as the sample has not been established yet. That is, factors predicting onset of cerebral infarction, cardiac infarction, obstructive arteriosclerosis and the like which are critical and irreversible tissue damages subsequent to the metabolic syndrome have not been established to date.

Oxidative stress is thought to be one of the causes for many disorders including lifestyle-related diseases, and aging. Oxidative stress is also known to act as a promoting factor for vascular aging. As an oxidative stress marker, immunological determination of protein carbonylation and a TBAS method for determining lipoperoxidation are well known conventionally. However, these methods involve problems associated with specificity and reproducibility when serum is used as the sample. Meanwhile, in recent years, methods using s a specific antibody against oxidative denaturation of lipid and protein fractions in LDL have been developed. The oxidative denaturation shows a process in which proteins and lipids undergo irreversible denaturation and degradation and is known to be associated with the advancement of atherosclerosis. However, the oxidative denaturation may not be considered in part as an index of earlier indication of vascular aging.

A thiol group of proteins is oxidatively modified in a nonenzymatic fashion by oxidative stress, which causes intramolecularly cross-linked protein and inactivation of enzyme proteins, thus proceeding to functional changes of proteins. A thiol group is sometimes oxidized to cause irreversible modification. S-glutathionylation (protein-SSG, glutathionylation) plays a role of protecting functions and structures of proteins from such an irreversible oxidative modification. In other words, hydrogen peroxide modifies a thiol group of a cysteine of a protein and promotes an irreversible denaturation process with sulfenic acid (—SOH) and further with sulfinic acid (—SO₂H) and sulfonic acid (—SO₃H). Modification of a thiol group with glutathione is a reversible change and is also regulated by concentrations of reduced glutathione (GSH) and oxidized glutathione (GSSG). This is referred to as “redox”. Redox is an important mechanism that is associated with the function or activity of living organisms or cells and controls cellular differentiation and proliferation, and cellular death. In other words, the oxidative modification of a thiol group of a protein indicates an imbalance in the redox state of a living organism. Failure of redox results in apoptosis of tissue cells, and further, remodeling, and is considered to be deeply involved in the mechanism of vascular aging.

Methods for measuring a thiol group of a protein modified with glutathione have been reported so far.
1) An immunological method using an anti-glutathionylated bovine albumin antibody (non-patent reference 1). This method involves problems associated with specificity and measurement sensitivity, and is not suited for practical application.
2) A method using a biotinylated glutathione S-transferase (non-patent reference 2). This is an application of the fact that the glutathione S-transferase recognizes a glutathionylated thiol group through an enzyme reaction, but this method involves many problems associated with sensitivity and specificity.

[non-patent reference 1] Heille, O P et al., Eur. J. Neurosci., vol. 6, p. 793-804 (1994)

[non-patent reference 2] Cheng, G et al., Archiv. Biochem. Biophys., vol. 435, p. 42-49 (2005)

### DISCLOSURE OF THE INVENTION

### Problems to be Solved by the Invention

An object of the present invention is to provide a biomarker useful for prediction of vascular aging and an examination method for a lesion resulting from vascular aging using the biomarker as an index.

### Means for Solving the Problems

The inventors of the present invention made intensive studies and examinations continuously in order to solve the above-mentioned problems, paid attention particularly to an apolipoprotein B100 localized in low density lipoprotein (LDL), identified the domain of a glutathionylated thiol group in advance, then produced a specific antibody against the site, and established a highly sensitive immunoassay to complete the present invention.

That is, the present invention provides the following:
[1] A predicting factor for vascular aging comprising an apolipoprotein B100 having a glutathionylated thiol group, or a part thereof which includes the glutathionylated region and consists of at least 10 amino acid residues,
[2] The predicting factor according to the above-mentioned [1], wherein the apolipoprotein B100 and a part thereof include at least an amino acid sequence represented by SEQ ID NO: 2, wherein the glutathionylated region is a thiol group of a cysteine in the amino acid sequence,
[3] A method for examining a lesion resulting from vascular aging comprising measurement of an apolipoprotein B100 having a glutathionylated thiol group using a biological sample,
[4] The method according to the above-mentioned [3], wherein the biological sample is blood,
[5] The method according to the above-mentioned [3] or [4], wherein the lesion resulting from vascular aging is accompanied by a disorder selected from the group consisting of diabetes mellitus, cerebrovascular disorder, cardiovascular disorder, obstructive arteriosclerosis, dementia, atherosclerosis, hypertension, obesity, and the like,
[6] An antibody specifically recognizing an apolipoprotein B100 having a glutathionylated thiol group, or a part thereof which includes the glutathionylated region, and consists of at least 10 amino acid residues,
[7] An antibody specifically recognizing glutathionylation of a thiol group of a cysteine in an amino acid sequence represented by SEQ ID NO: 2, and
[8] A diagnostic agent or a diagnostic kit for a lesion resulting from vascular aging comprising an antibody specifically recognizing an apolipoprotein B100 having a glutathionylated thiol group.

### Effects of the Invention

According to the present invention, since the correlation between an apolipoprotein B100 having a glutathionylated thiol group and a vascular lesion has been elucidated, an apolipoprotein B100 wherein a thiol group is modified or a part thereof can be used as a predicting factor for vascular aging. An examination method of the present invention using such a predicting factor as an index can make judgment briefly and with high accuracy that there is a lesion resulting from vascular aging or a risk for vascular aging, and is useful for diagnosis, follow-up, prognostic expectation, preclinical diagnosis, carrier diagnosis, or the like of a lesion resulting from vascular aging. Further, an antibody, and a diagnostic agent and a diagnostic kit comprising the antibody of the present invention can be used as a tool suited for the examination method of the present invention.

### BRIEF DESCRIPTION OF THE DRAWINGS

FIG. 1 is a drawing showing immunological measurement of a glutathionylated apolipoprotein B100 by SDS-slab gel electrophoresis. In (A), control serum from a healthy subject and serum from an ASO patient are used as samples, which are stained by an anti-apolipoprotein B100 antibody. Lane 1: Control serum +5 mM DTT, Lane 2: Patient serum +5 mM DTT, Lane 3: Control serum, Lane 4: Patient serum. In (B), control serum from a healthy subject and serum from an ASO patient are used as samples, which are stained by an anti-glutathionylated apolipoprotein B100 antibody. Lane 1: Control serum +5 mM DTT, Lane 2: Patient serum +5 mM DTT, Lane 3: Control serum, Lane 4: Patient serum. In (C), a glutathionylated apolipoprotein B100 produced in the control serum from a healthy subject is stained by an anti-apolipoprotein B100 antibody (upper line) and by an anti-glutathionylated apolipoprotein B100 antibody (lower line). Lane 1: 5% P-ME added, Lane 2: P-ME not added, Lane 3: 5 mM glutathione +0.5 mM hydrogen peroxide added, Lane 4: 10 mM glutathione +0.5 mM hydrogen peroxide added.

FIG. 2 is a drawing showing measurement of a glutathionylated apolipoprotein B100 in the serum from an ASO patient. (control (n=10) average 0.92±0.43; I (n=6) average 2.96±1.08; II (n=27) average 3.18±1.47; III, IV (n=18) average 3.97±1.73)

FIG. 3 is a drawing showing measurement of a glutathionylated apolipoprotein B100 in the serum from a diabetes patient. (control (n=10) average 1.07±0.18; no DM complication (n=12) average 0.91±0.41; DM, ASO(−) with other complication (n=52) average 2.24±1.90; DM, ASO(+) (n=26) average 2.71±1.49)

FIG. 4 is a drawing showing investigation of quantitative performance of an immune reaction using a purified glutathionylated apolipoprotein B100 as the antigen and using an anti-glutathionylated apolipoprotein B100 antibody. (A) is an electrophoresis photograph showing measurement results by the western blot method. (B) is a quantification graph showing the results of (A).

FIG. 5 is a drawing showing detection of a protein in the serum from a diabetes patient isolated by the immunoprecipitation method. (A) shows an anti-glutathionylated apolipoprotein B100 antibody and (B) shows an anti-apolipoprotein B100 antibody.

FIG. 6 is a drawing showing the thiol transferase activity in the serum from an ASO patient.

### BEST MODE FOR CARRYING OUT THE INVENTION

The present invention provides a novel predicting factor for vascular aging. The present invention targets a region that underwent reversible oxidative modification, rather than a modified protein that changes irreversibly by “oxidative stress” considered to particularly accelerate vascular aging. Living organisms have a high reduction power and thus protect themselves from oxidative stress. A sensor for such stress is, for example, a thiol group of a protein. Thus, the present invention takes note of the thiol group in blood protein and attempts to measure its modification.

An apolipoprotein B100 is a protein constituting a low-density lipoprotein (LDL), and in human beings, for example, it is a protein consisting of an amino acid sequence represented by SEQ ID NO: 1. Oxidative stress causes, in an apolipoprotein B100, irreversible modification to become a denatured LDL, which is taken into vascular endothelial cells or vascular macrophage by a scavenger receptor expressed on cellular surfaces of such cells, forms plaque in the blood vessel and thereby contributes to the development of an arteriosclerotic lesion. Further, oxidative stress causes diversified lesions including vascular aging. As will be described in examples shown later, a significant increase in apolipoprotein B100 having a glutathionylated thiol group was observed in the serum from a diabetes patient with a vascular lesion as compared to the protein in the serum from a diabetes patient without a vascular lesion. Therefore, an apolipoprotein B100 having a glutathionylated thiol group can be an excellent predicting factor for vascular aging.

The predicting factor of the present invention is characterized in that it comprises an apolipoprotein B100 having a glutathionylated thiol group, or a part thereof which includes the glutathionylated region, and consists of at least 10 amino acid residues. Specifically, a polypeptide containing an amino acid sequence is preferred wherein at least a thiol group in the cysteine in the amino acid sequence represented by SEQ ID NO: 2 (VPSCKLDFRE) is glutathionylated.

“Glutathionylation of a thiol group” as used herein denotes a thiol group of a cysteine of an apolipoprotein B100 in a —S—SG state reversible from an ordinary reduced form (—SH) due to oxidative stress or the like. This is different from a state that is further irreversibly modified by oxidative stress.

Vascular aging includes a physiological change of a blood vessel (artery) due to increased age, and a pathological change of a blood vessel induced by hypertension, hyperlipidemia, obesity, diabetes mellitus, smoking, cirrhosis hepatis, auto immune disease, or the like.

Further, the present invention relates to an examination method of a lesion resulting from vascular aging characterized in that an apolipoprotein B100 having a glutathionylated thiol group is detected using a biological sample. The method of the present invention allows not only detection of a possibility of a lesion resulting from vascular aging, but also the risk of a lesion resulting from vascular aging.

In lesions resulting from vascular aging, arteriosclerotic changes such as damage of vascular endothelial cells, thrombus formation, intimal thickening, vascular blockage and plaque formation are recognized.

As disorders accompanied by lesions resulting from vascular aging, diabetes mellitus, cerebrovascular disturbance, cardiovascular disturbance, obstructive arteriosclerosis , dementia, atherosclerosis, hypertension, obesity and the like are mentioned.

Although a subject to which the examination method of the present invention can be applied is preferably a human being, mammals other than a human being may be used. For such mammals, for example, mouse, rat, rabbit, canine, feline, equine, sheep, bovine, goat, swine, miniature pig, hairless pig, simian and the like are mentioned.

A test subject of the present invention is not particularly limited, and for human beings, those undergoing general medical check-up, those having a vascular aging risk, or those with lesions resulting from vascular aging and the like may be mentioned.

“Those having a vascular aging risk” preferably denotes human beings and the like with diabetes mellitus, hypertension, hyperlipidemia, obesity, or metabolic syndrome.

A biological sample applicable to the method of the present invention is a tissue or a body fluid in which an apolipoprotein B100 can exist, and is preferably a blood sample. As for the blood, any of whole blood, serum, and plasma may be used, and these may be obtained properly by treating blood taken from a subject by an ordinary method. Although not particularly limited, a blood sample is preferably serum. An apolipoprotein B100 having a glutathionylated thiol group may exist in the form of an “LDL containing an apolipoprotein B100 having a glutathionylated thiol group (glutathionylated LDL)”, as well as an apolipoprotein B100 having a glutathionylated thiol group, while any of the forms can be measured by the method of the present invention.

In the present invention, detection of an apolipoprotein B100 having a glutathionylated thiol group in the blood sample isolated from a human being, is not particularly limited, and for example, methods such as an immunochemical method may be used. Among them, detection is preferably carried out by an immunochemical method that utilizes an antibody specifically recognizing an apolipoprotein B100 having a glutathionylated thiol group. Meanwhile, “detection of an apolipoprotein B100 having a glutathionylated thiol group” as used herein denotes not only qualitative detection of more than a certain concentration of an apolipoprotein B100 having a glutathionylated thiol group, but also quantitative measurement of the concentration of an apolipoprotein B100 having a glutathionylated thiol group.

There is no limitation for the immunochemical method used for measuring the concentration of an apolipoprotein B100 having a glutathionylated thiol group in a blood sample, and a conventionally known method such as dot-blot assay, western blot method, enzyme immunoassay (ELISA), latex agglutination assay, immuno-chromatography, radioimmunoassay (RIA), fluoroimmunoassay (FIA), and turbidimetry for measurement of turbidity accompanying the formation of an antigen-antibody complex are mentioned. Of them, it is preferable to determine the concentration of an apolipoprotein B100 having a glutathionylated thiol group from the difference of color development by the western blot method or dot-blot assay, while an antigen of a known concentration is used as the control.

When an apolipoprotein B100 having a glutathionylated thiol group is detected at more than a certain level in the blood sample, the subject from whom the blood sample is derived may be judged to have a lesion resulting from vascular aging or a risk for vascular aging. In this case, the higher the concentration of an apolipoprotein B100 having a glutathionylated thiol group in the blood sample, the higher the possibility or the risk that the subject has the lesion. Further, when an apolipoprotein B100 having a glutathionylated thiol group is detected at more than a certain level in the blood sample, it may be judged that the capability for reducing a glutathionylated apolipoprotein B100 in a living organism decreased in the subject from whom the blood sample is derived. In this case, the higher the concentration of an apolipoprotein B100 having a glutathionylated thiol group in a blood sample, the higher the possibility that the reducing power is decreased. On the other hand, when an apolipoprotein B100 having a glutathionylated thiol group is detected at less than a certain level in the blood sample, it may be judged that the subject from whom the blood sample is derived has a lower possibility or risk of having a lesion resulting from vascular aging, and that the capability of reducing a glutathionylated apolipoprotein B100 in living organisms is increased or maintained.

When an apolipoprotein B100 having a glutathionylated thiol group is detected in. the blood sample, and. when the amount of an apolipoprotein B100 having a glutathionylated thiol group in the blood sample is greater, it can be judged that a possibility or risk for having a lesion resulting from vascular aging is higher as compared to a case where the amount is less, and the possibility that the reducing power of a glutathionylated apolipoprotein B100 decreased in the living organism is higher.

Further, the present invention provides a diagnostic agent for a lesion resulting from vascular aging comprising a substance for detecting an apolipoprotein B100 having a glutathionylated thiol group.

As for the substance, although there is no limitation as long as the substance is capable of detecting an apolipoprotein B100 having a glutathionylated thiol group in the above-mentioned method, an antibody specifically recognizing an apolipoprotein B100 having a glutathionylated thiol group (hereafter, referred to as the “antibody of the present invention”) is preferable.

The “antibody” in the present invention includes natural antibodies such as a polyclonal antibody and a monoclonal antibody (mAb), chimeric antibodies that can be produced using a genetic recombination technology, humanized antibodies and single-chain antibodies, human antibodies that can be produced using a human antibody producing transgenic animal or the like, antibody fragments produced by Fab expression library, and binding fragments thereof, but is not limited thereto.

The antibody of the present invention included in the diagnostic agent may be either a polyclonal antibody or a monoclonal antibody as long as it specifically binds to an apolipoprotein B100 wherein a thiol group derived from a mammal, preferably from a human being, is glutathionylated.

The binding fragment denotes a region of a part of the above-mentioned antibody, and specifically, for example, F(ab′) ₂, Fab′, Fab, Fv (variable fragment of antibody), sFv, dsFv (disulphide stabilized Fv), dAb (single domain antibody) and the like are mentioned (Exp. Opin. Ther. Patents, Vol. 6, No. 5, p. 441-456, 1996).

The class of the antibody is not particularly limited and includes antibodies having any isotype such as IgG, IgM, IgA, IgD, IgE and the like. Preferably, IgG and IgM are mentioned, while IgG is more preferable when ease of purification and the like is taken into consideration.

A polyclonal antibody or a monoclonal antibody can be manufactured by an existing ordinary manufacturing method. That is, for example, an immunogen is given together with the Freund's Adjuvant as necessary to a mammal, e.g., mouse, rat, hamster, guinea pig, rabbit, feline, canine, swine, goat, equine or bovine, preferably mouse, rat, hamster, guinea pig, goat, equine or rabbit (in the case of polyclonal antibody) for immunization, and the same is given to mouse, rat or hamster (in the case of monoclonal antibody).

The immunogen is used cross-linked to a carrier, as necessary. For carriers, for example, BSA, KLH, and the like are mentioned. Further, a protein derived from animals to be immunized (e.g., a serum protein and the like) may be used as the carrier.

As for the immunogen used for production of an antibody of the present invention, an amino acid sequence represented by SEQ ID NO: 1 or a polypeptide having a partial sequence thereof is used. Preferably, the polypeptide comprises an amino acid sequence in which the thiol group of the cysteine in SEQ ID NO: 2 (VPSCKLDFRE) is glutathionylated or a partial sequence thereof. Specifically, the immunogen to be used for production of the antibody of the present invention is a peptide consisting of an amino acid sequence represented by SEQ ID NO: 2 (VPSCKLDFRE) or a partial sequence thereof. Although the length of the partial sequence is not limited as long as it possesses immunogenicity as an epitope, the length is preferably not less than 6 amino acids, more preferably not less than 8 amino acids, further preferably 10 amino acids.

For the sake of cross-linking the above-mentioned peptide to a carrier, one or a plurality of amino acids may be added. Although the number of amino acids to be added is not particularly limited, in consideration of the specificity of the antibody to be produced, the number of amino acids is preferably 1 to 10, more preferably 1 to 5, further preferably 1 to 2, most preferably 1.

Although the position of the amino acid to be added may be either an N-terminal or a C-terminal of the polypeptide, the amino acid is preferably added to an N-terminal.

Although the type of the amino acid to be added may be any of 20 types of amino acids known per se, preferably, at least one cysteine is included in the amino acid to be added. More preferably, the amino acid to be added consists of a cysteine.

Specifically, the polyclonal antibody can be produced as follows: that is, an immunogen is injected one to several times to a mouse, a rat, a hamster, a guinea pig, a goat, a horse or a rabbit, preferably a goat, a horse or a rabbit, more preferably a rabbit through subcutaneous, intramuscular, intravenous, foot pad, or intraperitoneal routes, and the mammal is sensitized. Normally, immunization is performed once to five times approximately every 1 to 14 days. after the first immunization, and serum is obtained from the mammal that is sensitized about 1 to 5 days after the final immunization.

While the serum may be used as the polyclonal antibody, preferably, the antibody is isolated and/or purified by saturated ammonium sulfate, an euglobulin sedimentation method, a caproic acid method, a caprylic acid method, ion-exchange chromatography (DEAE or DE52, etc.), anti-immunoglobulin column or protein A/G column, or affinity column chromatography using a column or the like in which an immunogen is cross-linked.

A monoclonal antibody is produced by preparing a hybridoma (fused cell) from the antibody-producing cell obtained from the above-mentioned sensitized animal and a myeloma cell without auto-antibody producibility (myeloma cell), cloning the hybridoma, and selecting a clone that produces a monoclonal antibody exhibiting specific affinity to the immunogen used for immunization of the mammal.

Specifically, the monoclonal antibody can be produced as follows: that is, a mouse, a rat or a hamster (including transgenic animals created so as to produce an antibody derived from other animals such as a human antibody producing transgenic mouse) is sensitized by one to several times of injections of the immunogen through subcutaneous, intramuscular, intravenous, foot pad, or intraperitoneal routes or by grafting the same. Normally, immunization is performed once to four times approximately every 1 to 14 days after the first immunization, and antibody-producing cells are obtained from the mammal that is sensitized about 1 to 5 days after the final immunization.

Preparation of a hybridoma (fused cell) that secretes a monoclonal antibody can be performed according to the method proposed by Kohler and Millstein (Nature, Vol. 256, p. 495-497, 1975) and a modification method conforming to the method. In other words, antibody-producing cells contained in the spleen, lymph node, bone marrow or tonsilla, or the like obtained from the sensitized mammal as mentioned above, preferably antibody-producing cells contained in the spleen, and myeloma cells without auto-antibody producibility derived from preferably a mammal such as a mouse, a rat, a guinea pig, a hamster, a rabbit or a human being, more preferably from a mouse, a rat or a human being, are subject to cell fusion to prepare such a hybridoma.

For myeloma cells used for cell fusion, for example, myeloma P3/X63-AG8. 653 (653; ATCC No. CRL1580), P3/NSI/1-Ag4-1 (NS-1), P3/X63-Ag8.U1 (P3U1), SP2/0-Ag14 (Sp2/0, Sp2), PAI, F0 or BW5147, derived from a mouse, myeloma 210RCY3-Ag. 2. 3. derived from a rat, or myeloma U-266AR1, GM1500-6TG-A1-2, UC729-6, CEM-AGR, D1R11 or CEM-T15, derived from a human being, may be used.

Screening of hybridoma clones producing a monoclonal antibody can be performed by culturing hybridoma in, for example, a microtiter plate, and measuring the reactivity of the culture supernatant of the well where proliferation is observed, against the immunogen used in the above-mentioned sensitization by enzyme immunoassay, for example, RIA or ELISA and the like.

Production of a monoclonal antibody from the hybridoma can be performed by culturing the hybridoma in vitro, or in vivo in a mouse, a rat, a guinea pig, a hamster or a rabbit or the like, preferably a mouse or a rat, more preferably in vivo in peritoneal effusion and the like of a mouse, and isolating the antibody from the culture supernatant or peritoneal effusion of the mammal thus obtained.

When culturing is performed in vitro, the hybridoma is proliferated, maintained and stored to meet with various conditions such as characteristics of the cell species to be cultured, the purpose of the test and research, and the culture method, and a known culture medium such as those used for production of a monoclonal antibody in the culture supernatant or any culture medium induced and prepared from a known basal medium can be used.

The monoclonal antibody is preferably isolated and/or purified similarly as the above-mentioned polyclonal antibody.

A chimeric antibody can be produced by reference to, for example, “Experimental Medicine (Extra edition), Vol. 6, No. 10, 1988”, JP-B-H03-73280, a humanized antibody can be produced by reference to, for example, JP-T-H04-506458, JP-A-62-296890, and a human antibody can be produced by reference to, for example, Nature Genetics, Vol. 15, p. 146-156, 1997, Nature Genetics, Vol. 7, p. 13-21, 1994, JP-T-H04-504365, W094/25585, “Nikkei Science, June, pp 40-50, 1995”, Nature, Vol. 368, p. 856-859, 1994, JP-T-H06-500233, respectively.

F(ab′)₂ and Fab′ can be produced by treating immunoglobulin with pepsin and papain, respectively, which are proteolytic enzymes.

The antibody of the present invention produced by the above-mentioned method is able to recognize a human apolipoprotein B100 having a glutathionylated thiol group with extremely high sensitivity and specificity and is therefore very useful for detection and quantitative determination of an apolipoprotein B100 having a glutathionylated thiol group in a biological sample derived from a human.

The antibody of the present invention is included in the diagnostic agent in a free state, a labeled state, or a solid-phased state.

Use of the diagnostic agent of the present invention allows diagnosis of lesions resulting from vascular aging by the above-mentioned methods.

The diagnostic agent of the present invention can also be used as a diagnostic kit of lesions resulting from vascular aging which further comprises a reagent or the like used in the above-mentioned detection method. For the reagent or the like, specifically, a buffer solution for dilution of reagents and biological samples, a fluorescence dye, a reaction vessel, a positive control, a negative control, an instruction manual describing the inspection protocol and the like are mentioned. These elements may be mixed in advance as necessary. Use of the kit allows simple diagnosis of lesions resulting from vascular aging according to the present invention.

Contents of all publications including patent specifications cited herein are incorporated herein by reference in their entirety as if they had each been set forth in full.

Although the present invention will be described hereafter in detail referring to examples, the present invention is not limited to the following examples. In the examples shown hereafter an “apolipoprotein B100 having a glutathionylated thiol group” will be abbreviated as a “glutathionylated apolipoprotein B100”.

### EXAMPLES

Apolipoprotein B100 protein C-terminal 10 amino acid residues (VPSCKLDFRE: SEQ ID NO.: 2) containing a reactive thiol group were synthesized (SIGMA). Glutathione (100 mM) was added to the synthesized peptide solution, which was then incubated at 37° C. for 15 minutes in the presence of hydrogen peroxide (0.5 mM) for glutathionylation of the thiol group. The glutathionylation disappeared by reduction by treatment with a reducing agent and therefore, it was confirmed that the thiol group was in the state of becoming reversible —S—SG (glutathionylated). A specific antibody against a peptide having a glutathionylated thiol group was produced in a house rabbit (see Susanne Mohr et al., The Journal of Biological Chemistry, vol. 274(14), p. 9427-9430 (1999), Hjelle O. P. et al., Eur. J. Neurosci., vol. 6(5), p. 793-804 (1994) and Cheng G et al., Archives of Biochemistry and Biophysics, vol. 435, p. 42-49 (2005)).

### “Quantitative Determination of Glutathionylated Apolipoprotein B100 in Serum”

SDS-slab gel electrophoresis was performed in the absence of a reducing agent such as beta-mercaptoethanol (β-ME) or DTT (dithiothreitol) using human serum as a sample. It was stained with an anti-glutathionylated apolipoprotein B100 antibody and an anti-apolipoprotein B100 antibody using the western blot method and the degree of glutathionylation of the thiol group was determined.

### Results of Experiments

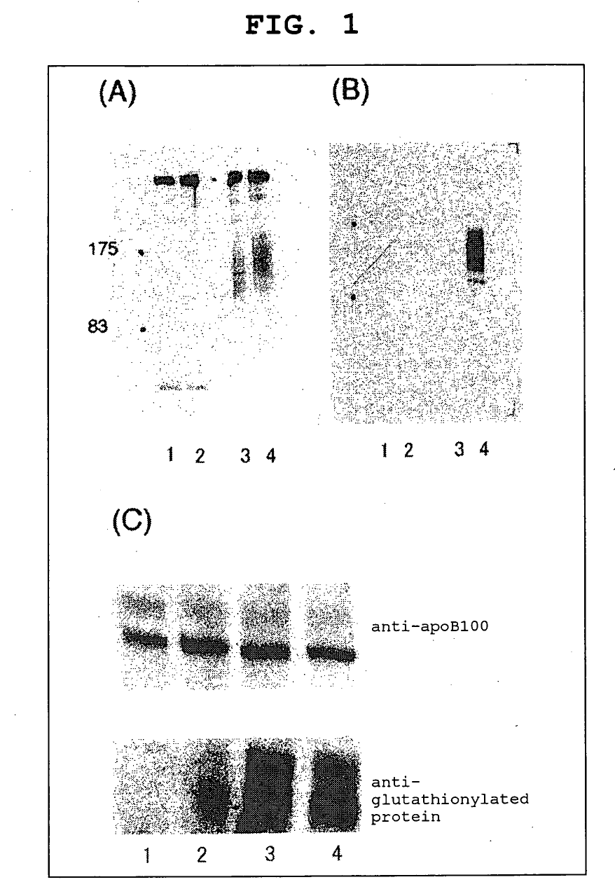

### 1. Immunological Measurement by SDS-slab Gel Electrophoresis (FIG. 1)

(A) Control serum from a healthy subject and serum from a patient with obstructive arteriosclerosis (ASO) were used as samples and stained with an anti-apolipoprotein B100 antibody (provided by SRL Ltd.). Bands corresponding to an apolipoprotein B100 were recognized only in the absence of DTT (lanes 3 and 4). The degree of staining was slight in the healthy control (lane 3) and was strong in the sample from an ASO patient (lane 4).

(B) The same samples were stained with an anti-glutathionylated apolipoprotein B100 antibody. They were stained with an anti-glutathionylated apolipoprotein B100 antibody in the absence of DTT. Although glutathionylation of the thiol group was observed with the fragmented apolipoprotein B100 fraction of an ASO patient serum, glutathionylation occurred also in a normal size apolipoprotein B100 that was not fragmented (lane 4). Meanwhile, it is known that an apolipoprotein B100 is fragmented during the course of oxidative denaturation due to oxidative stress and that lipid peroxides also increase (Cushing SD et al., Proc. Natl. Acad. Sci. USA, vol. 87, p. 5134-5138 (1990)).

(C) Preparation of glutathionylated apolipoprotein B100 using control serum from healthy subject

Hydrogen peroxide (0.5 mM) and glutathione (5 mM, 10 mM) were added to the control serum in the absence of β-ME, and reacted at 37° C. for 24 hours. Then, presence of a glutathionylated apolipoprotein B100 that was not identified in the control was observed (lanes 3 and 4). The result thus obtained indicates that the thiol group of an apolipoprotein B100 is glutathionylated by oxidative stress.

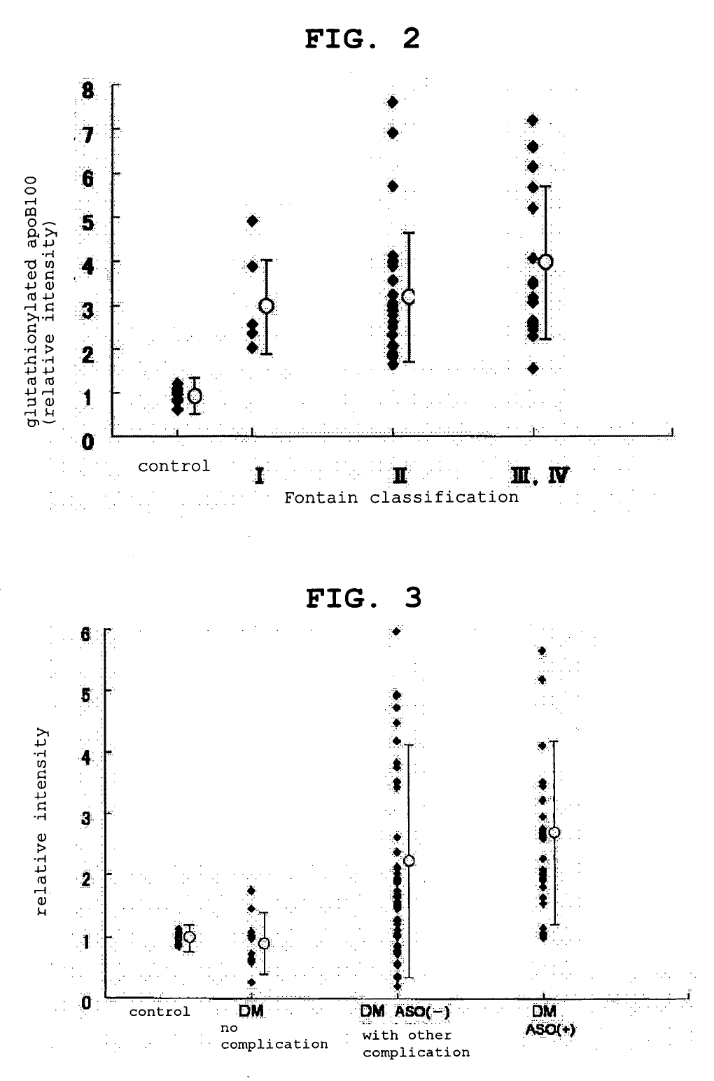

2. Measurement of Glutathionylated Apolipoprotein B100 in Serum from ASO Patient (FIG. 2)

Glutathionylated apolipoprotein B100 was measured using serum from an ASO patient as a sample and a significant increase in glutathionylated apolipoprotein B100 was observed with serum from an ASO patient as compared to the control serum from a healthy subject (p<0.001). Pathological conditions of an ASO patient were investigated according to the Fontain classification, which revealed that there was no lo significant correlation, although an upward tendency of a glutathionylated apolipoprotein B100 was observed with the progress of the disease condition.

The results thus obtained suggest that determination of the concentration of a glutathionylated apolipoprotein B100 in the serum using the antibody of the present invention allows discrimination between healthy people and early-stage ASO patients.

### 3. Investigation of Diabetic Patients (FIG. 3)

Glutathionylated apolipoprotein B100 in diabetic patients (DM) was investigated with regard to presence or absence of a vascular lesion. A significantly correlative increase in glutathionylated apolipoprotein B100 was observed with the serum from diabetic patients with a vascular lesion or highly liable to have the same in the future as compared to the serum from diabetic patients without a vascular lesion or less liable to have the same in the future (Table 1).

TABLE 1
Significant difference (t-test)

| | | DM, ASO(−) | |
|---|---|---|---|
| | DM | (with other | |
| | (no complication) | complication) | DM, ASO(+) |
| control | 0.467 | 0.0000208* | 0.00000441* |
| DM (no | | 0.0000227* | 0.00000271* |
| complication) | | | |
| DM, ASO(−) | | | 0.239 |
| (with other | | | |
| complication) | | | |

(Numerical value shows probability of no significant difference.
*p < 0.05)

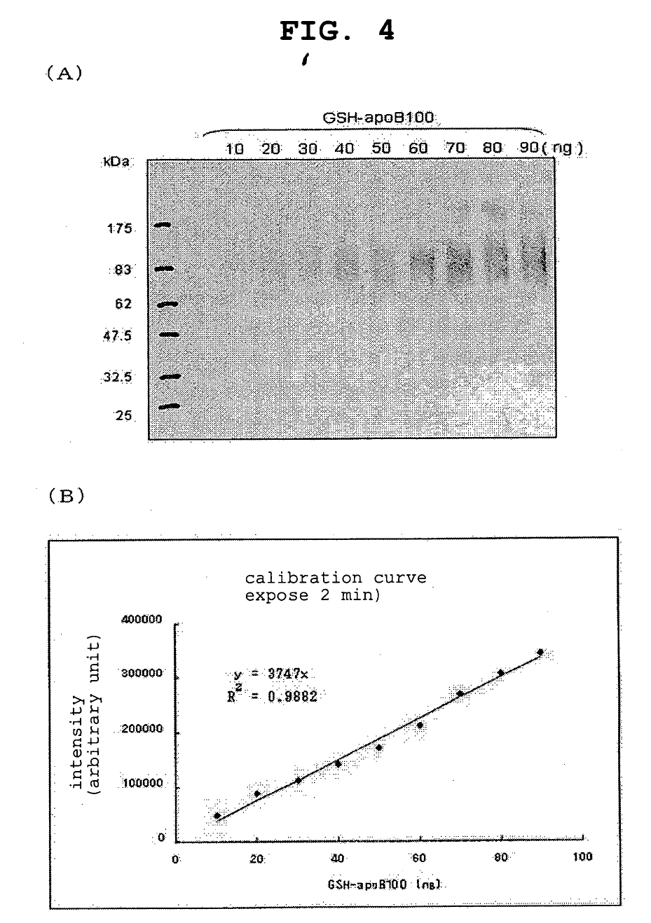

### 4. Quantitative Performance of Anti-glutathionylated Apolipoprotein B100 Antibody and Antigen (Purified Glutathionylated Apolipoprotein B100) in Immune Reaction (FIG. 4)

### (A) Quantitative Determination of Purified Glutathionylated Apolipoprotein B100 by Western Blot Method Using Anti-glutathionylated Apolipoprotein B100 Antibody

A glutathionylated apolipoprotein B100 (GSH-apoB100) equivalent to 10 ng to 90 ng was subjected to electrophoresis and was assayed by the western blot method. An anti-glutathionylated apolipoprotein B100 antibody was diluted 5000-fold to be used as the antibody and was reacted at room temperature for 1 hour. Intensive bands were observed at locations corresponding to glutathionylated apolipoprotein B100 fractions.

### (B) Analysis of Data of (A)

The results of (A) were quantified to obtain a calibration curve. As a result, a calibration curve showing antigen-level-dependent linearity was obtained.

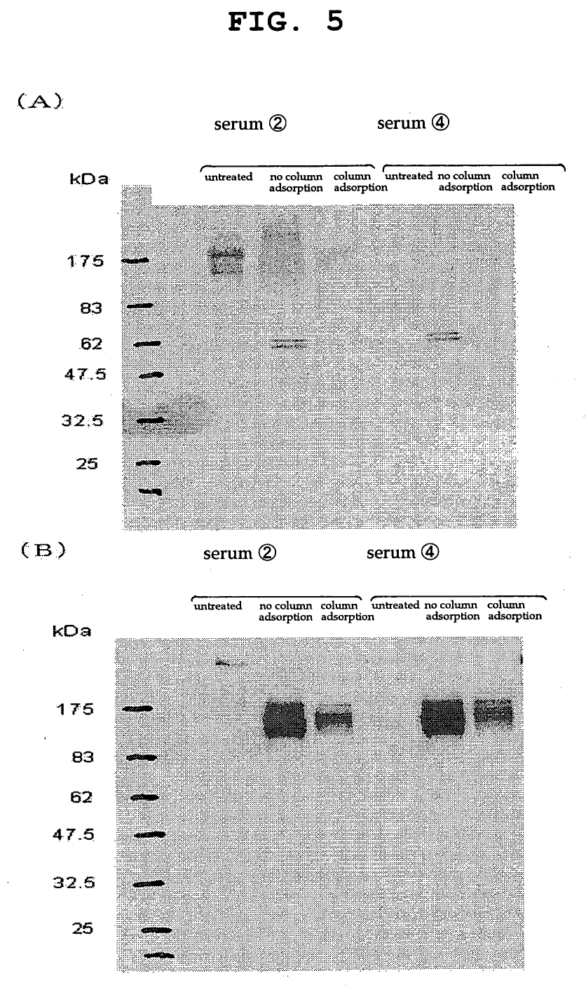

5. Extraction of Protein that Reacts with Anti-apolipoprotein B100 Antibody from Patient Serum Using Immunoprecipitation Method and Confirmation of Effects by Causing Anti-glutathionylated Apolipoprotein B100 Antibody to Act Thereon (FIG. 5)

The serum from patients and an anti-apolipoprotein B100 antibody (SRL Ltd.) were caused to react and a protein that reacts with an anti-apolipoprotein B100 antibody was separated and extracted using an immunoprecipitation method using protein A sepharose. Following this, each of original serum (untreated), a protein not reacted with an anti-apolipoprotein B100 antibody (not adsorbed by column), and a protein bound to an anti-apolipoprotein B100 antibody (adsorbed by column) was subjected to electrophoresis, and an anti-glutathionylated apolipoprotein B100 antibody (FIG. 5 (A)) and an anti-apolipoprotein B100 antibody (FIG. 5 (B)) were caused to act. The protein immunoprecipitated by an anti-apolipoprotein B100 antibody was detected around 175 kDa for both anti-glutathionylated apoliprotein B100 antibody and anti-apolipoprotein B100 antibody, which were approximately correspondent to each other. This finding revealed that a band detected by an anti-apolipoprotein B100 antibody is specifically detected by an anti-glutathionylated apolipoprotein B100 antibody.

From the results thus obtained, it has been found that the measurement method using the antibody of the present invention has excellent sensitivity and specificity as compared to the conventional methods. If a measurement method as a stress sensor is established, extensive clinical applications as a novel vascular aging marker would become possible.

6. Provision of Predicting Factor for Vascular Aging and of Examination Method of Early-stage Lesion Resulting from Vascular Aging

The meaning of measurement of a glutathionylated apolipoprotein B100 as a risk factor for obstructive arteriosclerosis was investigated.

Using serum from 41 patients with obstructive arteriosclerosis and from 38 controls, a glutathionylated apolipoprotein B100 was measured and investigated by a Yates continuity-corrected χ² test (Table 2).

It is apparent that a glutathionylated apolipoprotein B100 is an assessment factor having both extremely high sensitivity and specificity as compared to hs-CRP value that is already known to elevate in this disorder, and soluble LOX-1 (sLOX-1) value that is appreciated as an early diagnosis marker for vascular endothelial cell disorders such as cardiac infarction.

TABLE 2
Sensitivity and specificity of sLOX-1, hs-CRP,
and S-glutathionylated protein for ASO patients

| | | |
|---|---|---|
| | | S-glutathionylated |
| sLOX-1 | hs-CRP | protein |

Control (n = 38)

| | | | |
|---|---|---|---|
| Positive, n | 21 | 13 | 4 |
| Specificity | 45 | 66 | 89 |

ASO (n = 41)

| | | | |
|---|---|---|---|
| Positive, n | 31 | 18 | 28 |
| χ² | 2.78 | 0.42 | 24.96 |
| P | 0.095 | 0.515 | <0.0001 |
| Sensitivity | 76 | 44 | 68 |

Cut-off levels are 100 pg/ml for sLOX-1, 4 μl/ml for hs-GRP, and 2.5 relative intensity for thiol-modification. χ² was determined by a Yates continuity-corrected χ² test, and the probability value was obtained from comparison with non-ASO patients.

### 7. Meaning as Index for Decrease of Blood Redox Ability Which is the Basis of Vascular Aging

Investigation was made as to what a glutathionylated apolipoprotein B100 reflects in a living organism.

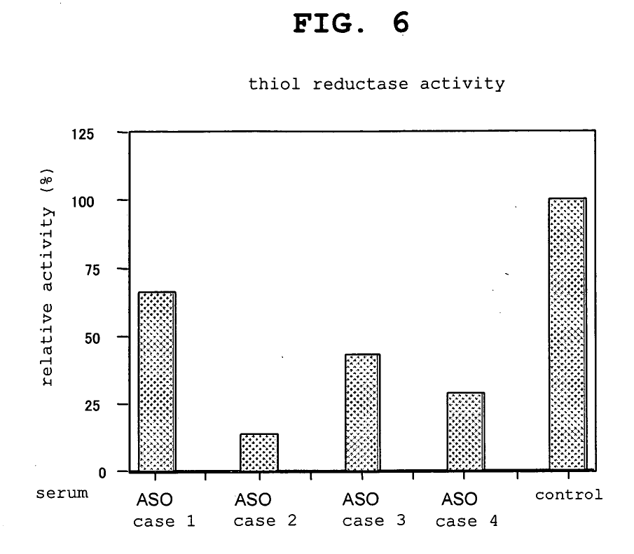

In the serum, vitamins which are antioxidative substances, glutathione, glutathione peroxidase, thioredoxin and glutaredoxin (GRX), which are redox proteins, and the like are present. Although these substances are considered to execute indirectly reduction of a glutathionylated serum protein, they do not directly reduce glutathionylated serum protein. The present inventors focused attention on GRX that has a direct reduction action on a cysteine group oxidatively modified in a glutathionylated protein, and have developed a method for measuring the activity of GRX dependent thiol transferase in the serum using low molecular weight protein tyrosine phosphatase (LMW-PTP) (Kanda, M, Kondo, T. et al., J. Biol. Chem., vol. 281(39), p. 28518-28528, 2006) which is a tyrosine phosphatase discovered by the present inventors to be a substrate of GRX. Specifically, LMW-PTP (100 μl/50 μg (2.8 nmols)) and ³⁵S-GSH (5 μl (2 pmoles, 50 nmols DTT)) were mixed and placed on ice for 5 minutes, then GSSG (28 nmoles) and H₂O₂ (100 nmols) (total amount 150 μl) were added thereto and the mixture was left standing at 4° C. for 24 hours and filtrated by spin column to yield ³⁵S-GS-LM-PTP (PTP labeled) . ³⁵S-GS-LM-PTP (5 μl) thus obtained and serum (0.5 μl) were mixed, 0.2 M NaH₂PO₄ (1 μl) (adjust pH to 7.0) and 7.5 mM GSH or PBS (−) (1 μl) were added thereto and left at 37° C. for 30 minutes, a 2×laemmli's solution (7.5 μl) was added to obtain a measurement mixture, and were subjected to the measurement of radioactivity released from a cysteine group of radiolabeled glutathionylated LMW-PTP. As a result, all patients with obstructive arteriosclerosis showed reduced activity of GRX dependent thiol transferase in the blood as compared to that of the control (FIG. 6). This suggests that the reduction power decreases in the patient serum, a protein SH group is in a state easily modified with oxidative stress, and that a power for reducing those oxidatively modified (glutathionylated) is low.

### 8. Prediction of Vascular Lesion Progression Unrecognizable by Other Measurement Methods

### Assessment of Glutathionylated Apolipoprotein B100 as Vascular Lesion Predicting Factor

Although definite diagnosis of obstructive arteriosclerosis by angiography after progression of the lesion has been developed, prediction of an early stage of onset and incipient stage is not at all sufficient. An ABI inspection is objectively assessed at an early stage. In patients with obstructive arteriosclerosis, an extremely high positive correlation is observed between the ABI level and the glutathionylated apolipoprotein B100 level. Further, determination of the glutathionylated apolipoprotein B100 level of serum from patients with diabetes mellitus, which is one of the incipient disorders of this disease, revealed elevation of the glutathionylated apolipoprotein B100 level even with those who did not show any apparent symptom or change in the ABI level. They are considered to be in incipient stage and require careful follow-up in the future (data not shown). Meanwhile, though in one case, the glutathionylated apolipoprotein B100 level of a diabetic patient who complained of intermittent pain in the lower legs was measured and found to be normal. In this case, the patient had a complication of marked hypertension, obesity, and hyperlipidemia, and was strongly suspected of suffering obstructive arteriosclerosis. However, since the ABI level was also normal, the possibility of other disorder was strongly suggested. From this incident, it is expected that a more accurate diagnosis will be enabled by determination of the glutathionylated apolipoprotein B100 level in addition to the sole determination of ABI.

### INDUSTRIAL APPLICABILITY

It is considered that a method of determining, as a stress sensor, a serum apolipoprotein B100 protein having a glutathionylated thiol group can be extensively used in clinical applications as a novel vascular aging marker. Metabolic syndrome patients constantly increasing in number year by year may encounter serious lifestyle-related diseases, which include cerebral infarction, cardiac infarction, obstructive arteriosclerosis and the like due to vascular aging. In addition, a vascular lesion underlies a complication of diabetes mellitus such as blindness and impairment of renal function. Factors for predicting onset of these disorders have not been established so far. It is expected that the method of determining a vascular aging marker established by the present invention would make great contribution to determination of prognosis of metabolic syndrome patients as well as diabetic patients. Further, development of a screening system for anti-arteriosclerotic agents using this probe is also possible. It is also expected that the determination method of the present invention would be applicable to objective diagnosis and judgment of therapeutic effects of dementia which is supposed to become a serious social problem in the future.

This application is based on a patent application No. 2005-344630 filed in Japan (filing date: Nov. 29, 2005), the contents of which are incorporated in full herein by this reference.

1. A predicting factor for vascular aging comprising an apolipoprotein B100 having a glutathionylated thiol group, or a part thereof which includes the glutathionylated region and consists of at least 10 amino acid residues.

2. The predicting factor of claim 1, wherein the apolipoprotein B 100 and a part thereof include at least an amino acid sequence represented by SEQ ID NO: 2, wherein the glutathionylated region is a thiol group of a cysteine in the amino acid sequence.

3. A method for examining a lesion resulting from vascular aging comprising measurement of an apolipoprotein B 100 having a glutathionylated thiol group using a biological sample.

4. The method of claim 3, wherein the biological sample is blood.

5. The method of claim 3, wherein the lesion resulting from vascular aging is accompanied by a disorder selected from the group consisting of diabetes mellitus, cerebrovascular disorder, cardiovascular disorder, obstructive arteriosclerosis, dementia, atherosclerosis, hypertension, obesity, and the like.

6. An antibody specifically recognizing an apolipoprotein B100 having a glutathionylated thiol group, or a part thereof which includes the glutathionylated region, and consists of at least 10 amino acid residues.

7. An antibody specifically recognizing glutathionylation of a thiol group of a cysteine in an amino acid sequence represented by SEQ ID NO: 2.

8. A diagnostic agent or a diagnostic kit for a lesion resulting from vascular aging comprising an antibody specifically recognizing an apolipoprotein B 100 having a glutathionylated thiol group.

9. The method of claim 4, wherein the lesion resulting from vascular aging is accompanied by a disorder selected from the group consisting of diabetes mellitus, cerebrovascular disorder, cardiovascular disorder, obstructive arteriosclerosis, dementia, atherosclerosis, hypertension, obesity, and the like.
